(19) United States
(12) Patent Application Publication
(10) Pub. No.: US 2017/0156615 A1
(43) Pub. Date: Jun. 8, 2017

(54) DISPOSABLE HEART MONITORING SYSTEM, APPARATUS AND METHOD

(71) Applicants: Reza Shirazi, San Diego, CA (US); Arshia Noori, Los Angeles, CA (US)

(72) Inventors: Reza Shirazi, San Diego, CA (US); Arshia Noori, Los Angeles, CA (US)

(21) Appl. No.: 14/961,254

(22) Filed: Dec. 7, 2015

(51) Int. Cl. A61B 5/0408; A61B 5/0205; A61B 5/1455; A61B 5/00

(52) U.S. Cl. CPC A61B 5/0408; A61B 5/6833; A61B 5/0205; A61B 5/14551

(57) ABSTRACT

A disposable, heart monitoring device is disclosed. The device comprises a disposable, wearable membrane comprising an adhesive surface and electrodes disposed within or on the membrane. The membrane is formed such that some of the electrodes are positioned on a user's chest over locations corresponding to chest electrode positions of a standard electrocardiogram, while other electrodes, corresponding to peripheral electrodes of a standard electrocardiogram, are also attached to the membrane for placement against the user's chest. The device further comprise a signal collection unit coupled to the electrodes for receiving electrical signals from the electrodes, and means for providing the electrical signals to an external processing device for analyzing the electrical signals to evaluate one or more attributes of the user's heart.

### BACKGROUND

I. Field of Use

The present application relates to the field of medical devices. More specifically, the present invention relates to a system, apparatus and method for allowing heart patients to perform heart monitoring procedures outside a typical medical center setting.

II. Description of the Related Art

Heart monitoring procedures, such as electrocardiagrams have been in use for many years, allowing doctors to record electrical activity of the heart over a period of time using electrodes placed on a patient's body. These electrodes detect tiny electrical changes on the skin that arise from the heart muscle depolarizing during each heartbeat.

In a conventional 12 lead ECG, ten electrodes are placed on the patient's limbs and on the surface of the chest. The overall magnitude of the heart's electrical potential is then measured from twelve different angles (“leads”) and is recorded over a period of time (usually 10 seconds). In this way, the overall magnitude and direction of the heart's electrical depolarization is captured at each moment throughout the cardiac cycle. The graph of voltage versus time produced by this noninvasive medical procedure is referred to as an electrocardiogram (ECG). Trained physicians and cardiologists can use ECGs to recognize abnormalities in cardiac function and provide essential medical intervention and advice as needed.

The 12-lead electrocardiogram remains an essential tool for screening and diagnostic workup of a person's heart. It is the current gold standard for evaluating electrical properties of the heart and identifying life-threatening conditions such as myocardial infarction (heart attack) and arrhythmias (irregular heart rhythm). Ten electrodes are used for a 12-lead ECG. The electrodes usually consist of a conducting gel, embedded in the middle of a self-adhesive pad. The names and correct locations for each electrode are as follows:

| Electrode | |
|---|---|
| Name | Electrode Placement |
| RA | On the right arm, avoiding thick muscle |
| LA | In the same location where RA was placed, but on the left |
| | arm. |
| LA | On the right leg, lateral calf muscle. |
| LL | In the same location where RL was placed, but on the left leg. |
| V₁ | In the fourth intercostal space (between ribs 4 and 5) just to |
| | the right of the sternum (breastbone). |
| V₂ | In the fourth intercostal space (between ribs 4 and 5) just to |
| | the left of the sternum. |
| V₃ | Between leads V₂ and V₄. |
| V4 | In the fifth intercostal space (between ribs 5 and 6) in the mid- |
| | clavicular line. |
| V₅ | Horizontally even with V₄, in the left anterior axillary line. |
| V₆ | Horizontally even with V₄ and V₅ in the midaxillary line. |

The term “lead” in electrocardiography refers to the 12 different vectors along which the heart's depolarization is measured and recorded. There are a total of six limb leads and augmented limb leads arranged like spokes of a wheel in the coronal plane (vertical) and six precordial leads that lie on the perpendicular transverse plane (horizontal).

Each of these leads represents the electrical potential difference between two points. For each lead, the positive pole is one of the ten electrodes. In bipolar leads, the negative pole is a different one of the electrodes, while in unipolar leads, the negative pole is a composite pole known as Wilson's central terminal. Wilson's central terminal VW is produced by averaging the measurements from the electrodes RA, LA, and LL to give an average potential across the body:

V_{W}=⅓(RA+LA+LL)

In a 12-lead ECG, all leads except the limb leads are unipolar (aVR, aVL, aVF, V₁, V₂, V₃, V₄, V₅, and V₆). Leads I, II and III are called the limb leads. The electrodes that form these signals are located on the limbs—one on each arm and one on the left leg. The limb leads form the points of what is known as Einthoven's triangle.

Lead I is the voltage between the (positive) left arm (LA) electrode and right arm (RA) electrode:

I=LA−RA

Lead II is the voltage between the (positive) left leg (LL) electrode and the right arm (RA) electrode:

II=LL−RA

Lead III is the voltage between the (positive) left leg (LL) electrode and the left arm (LA) electrode:

III=LL−LA

Leads aVR, aVL, and aVF are the augmented limb leads. They are derived from the same three electrodes as leads I, II, and III, but they use Wilson's central terminal as their negative pole.

Lead augmented vector right (aVR)′ has the positive electrode on the right arm. The negative pole is a combination of the left arm electrode and the left leg electrode:

aVR=RA−½(LA+LL)=3/2(RA−V_{W})

Lead augmented vector left (aVL) has the positive electrode on the left arm. The negative pole is a combination of the right arm electrode and the left leg electrode:

aVL=LA−½(RA+LL)=3/2(LA−V_{W})

Lead augmented vector foot (aVF) has the positive electrode on the left leg. The negative pole is a combination of the right arm electrode and the left arm electrode:

aVF=LL−½(RA+LA)=3/2(LL−V_{W})

The precordial leads lie in the transverse (horizontal) plane, perpendicular to the other six leads. The six precordial electrodes act as the positive poles for the six corresponding precordial leads: V₁, V₂, V₃, V₄, V₅, and V₆). Wilson's central terminal is used as the negative pole.

There are a number of drawbacks to prior art heart-monitoring procedures, however. It requires placement of 10 electrodes on a patient's body and an equal number of long wires that connect the electrodes to the ECG machine. The test must be performed by a trained technician for correct placement of the electrodes and to correctly connect of them to the ECG machine. At times, incorrectly placed electrodes or loose connections occur that result in inaccurate interpretation or suboptimal data quality. ECGs must be performed in a doctor's office or other medical facility, as the ECG machines are bulky, expensive and require a trained technician, as mentioned previously. Thus, patients must leave their homes to have an ECG performed. ECGs are expensive as well, due to the high cost of the ECG machine, the technician's time and a doctor's time to review the results.

In addition, the standard 12-Lead ECG only provides a 10 second snapshot in time of a patient's heart activity. Hence, it is only useful if an unknown arrhythmia is present at the time the ECG is being performed. The overwhelming majority of arrhythmias, however, in the clinical setting are not sustained and hence most arrhythmias go undetected for many years or sometimes never get diagnosed. In response to this, continuous event recorders and mobile telemetry units were created. These units allow one to wear a monitor for a continuous period of time of up to a month and sometimes for years (implantable versions). The limitation of these devices is that, at most, they provide 3 leads of data and the majority are limited to 2 or 1 useful lead. Although these devices enable continuous heart monitoring, they fail to provide enough sensory definition to accurately diagnose problems such as arrhythmia.

Thus, it would be desirable to allow patients to perform ECGs in their homes without the use of standard ECG machines and without a trained technician needed. This would reduce the cost of ECGs, encourage more use of ECGs, and provide a convenient way for patients to monitor their hearts.

### SUMMARY

The embodiments described herein relate to a system, apparatus, and method for home heart monitoring. In one embodiment, an apparatus is described, comprising a membrane comprising an adhesive surface for adhering the membrane to a human chest, a first plurality of electrodes attached to the membrane at locations corresponding to chest electrode positions of a standard heart monitoring procedure, a second plurality of electrodes attached to the membrane corresponding to peripheral electrodes of a standard electrocardiogram for placement against a human chest, a signal collection unit coupled to the first plurality of electrodes and the second plurality of electrodes for receiving electrical signals from the first plurality of electrodes and the second plurality of electrodes, and means for providing the electrical signals to a processing device separate from the disposable heart monitoring device for analyzing the electrical signals to evaluate one or more attributes of a human heart.

In another embodiment, a system is described, comprising a disposable membrane comprising an adhesive surface for adhering the membrane to a human chest, a plurality of electrodes; and a signal collection unit coupled to the plurality of electrodes for receiving electrical signals from the plurality of electrodes and for providing the electrical signals to an external processing device separate from the disposable heart monitoring device for analyzing the electrical signals to evaluate one or more attributes of a human heart. The external processing device comprises means for receiving the electrical signals from the signal collection unit, a memory for storing the electrical signals and processor-executable instructions, and a processor for executing the processor-executable instructions that cause the external processing device to compute one or more graphs of the electrical signals vs. time, store the one or more graphs in the memory, and transmit the one or more graphs to a remote location.

### BRIEF DESCRIPTION OF THE DRAWINGS

The features, advantages, and objects of the embodiments discussed herein will become more apparent from the detailed description as set forth below, when taken in conjunction with the drawings in which like referenced characters identify correspondingly throughout, and wherein:

FIG. 1 is an illustration of a disposable heart monitoring device, fixed onto a chest of an adult male;

FIG. 2 is a functional block diagram of one embodiment of a signal collection unit as shown in FIG. 1;

FIG. 3 is an illustration of one embodiment of a system for providing home heart monitoring;

FIG. 4 is a functional block diagram of one embodiment of the external processing device shown in FIG. 3; and

FIG. 5 is a flow diagram illustrating one embodiment of a method for providing home heart monitoring, performed by the disposable heart monitoring device shown in FIG. 1 and the external processing device shown in FIG. 3.

### DETAILED DESCRIPTION

The inventive concepts described herein relate to embodiments of a disposable heart monitoring system, apparatus and method for home use by heart patients. In one embodiment, a wearable, disposable membrane or “patch” is affixed to a patient's chest using some form of adhesive disposed on one surface of the membrane. The membrane comprises a plurality of electrodes used to generate electrical signals as the patient's heart beats, the signals corresponding to, in one embodiment, standard 12-lead ECG signals well known in the art. However, the four peripheral electrodes typically used in standard ECGs are not placed onto traditional areas of the body, such as the left and right arms and legs, but, rather, they are incorporated into the membrane and positioned over the chest, along with the other chest electrodes. The membrane further comprises a signal collection unit, coupled to the electrodes, for receiving the electrical signals produced by the electrodes, and either storing them for later analysis and/or providing them to a data analysis unit, such as a smart phone, which analyses the electric signals to generate an echocardiogram, e.g., one or more graphs illustrating typical, 12-lead ECG results, such as a combined P, QRS, and T waveform. Such a unique heart monitoring system allows for continuous heart monitoring, because users may wear the patch for long periods of time while heart functions are being recorded and/or monitored. The ability to obtain 12-lead accuracy on a continuous basis provides enough detailed information to doctors to differentiate, for example, a “wide-complex tachycardia” into whether is ventricular tachycardia or a supraventricular tachycardia with aberrant conduction, or differentiation between a “supraventricular tachycardia” into whether it is sinus tachycardia, atrial tachycardia, atrial flutter or an atrio-ventricular node dependent tachycardia. It may further allow differentiation of an “atrial flutter” into whether it is a typical counter-clockwise, clockwise or atypical atrial flutter. As a clinician, this type of differentiation is paramount as it dictates the type of treatment needed.

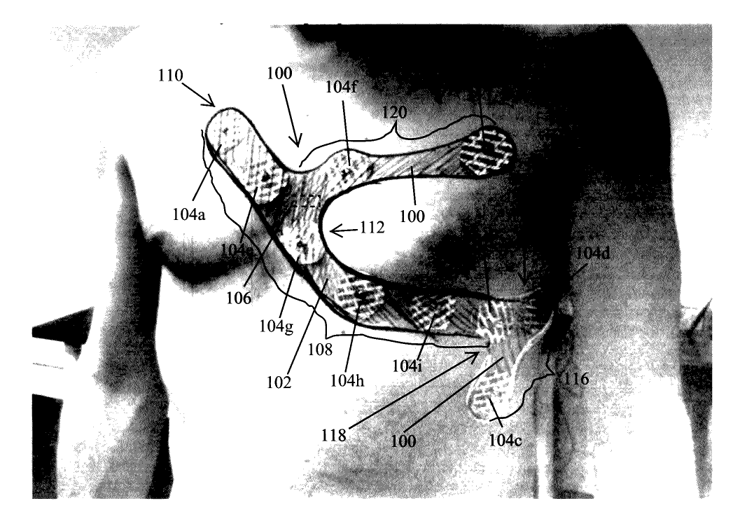

FIG. 1 is an illustration of a disposable heart monitoring device 100, fixed onto a chest of an adult male. The disposable heart monitoring device 100 comprises membrane 102, a number of electrodes 104a-104b, and a signal collection unit 106 coupled to the electrodes 104 to receive electronic signals generated by the electrodes as a heart inside the user's chest beats. As shown in FIG. 1, membrane 102 comprises an elongated, curved strip 108 having a first end 110 for placement over the user's upper right chest, a middle portion 112 for placement across the user's sternum, and a second end 114 for placement over the user's lower left chest. The elongated, curved strip 108 further comprising a short, lower strip 116 extending at an acute angle 118 from the second end 114, directed downward when the membrane 102 is positioned onto the user's chest, as shown. The elongated, curved strip 108 further comprises a mid-strip 120 extending approximately horizontally from the elongated, curved strip 108 for placement over an upper, left portion of the user's chest. In one embodiment, the elongated, curved strip 108, short, lower strip 116, and mid-strip 120 are manufactured as a single piece of material.

Membrane 102 can be made out one or more materials, including cloth, rubber, synthetic polymers or plastics, or just about any material that can adapt to the contours of a human chest. On one side of the membrane, an acrylate, such as methacrylates and epoxy diacrylates (which are also known as vinyl resins) are commonly used to adhere membrane 102 to the skin of a user's chest. The adhesive may be chosen to allow membrane 102 to remain on the user's skin for a short duration (e.g., for an hour) or it may be chosen so as to keep membrane 102 adhered to the skin for days or even weeks. In this embodiment, membrane 102 and the selected adhesive may be chosen to waterproof disposable heart monitoring device 100, so that a user may keep wearing disposable heart monitoring device 100 without having to remove it for showering.

The disposable heart monitoring device 100 may be manufactured in different sizes, to accommodate different body sizes, including varieties tailored to infants or small children. It may be of importance for user's to select a correct size of the disposable heart monitoring device 100, as the electrodes typically require accurate placement over various parts of the chest, and an incorrect size may result in sub-optimal electrode placement. In one embodiment, at least three sizes are contemplated for manufacture and sale, ranging in size from “small” to fit small children/infants, “medium” to fit older children, and “large” to fit most adults.

As mentioned above, the membrane 102 comprises a number of electrodes positioned at different locations along membrane 102. Such electrodes typically comprise a plastic substrate covered with a silver/silver chloride ionic compound to increase conductivity at the skin. The electrode is assembled with an electrolyte gel in which the principle anion is Cl−. Silver on the electrode surface oxidizes to silver ions in solution at the interface. These ions combine with Cl− already in solution to form the ionic compound Ag or AgCl. Today, nearly all biomonitoring electrodes used to monitor and record biopotentials are Ag/AgCl.

Electrodes 104 are typically fabricated by first making a plastic injection molded substrate and then coating it with a very thin layer of silver. The outer layer of the silver is converted to silver chloride. Various methods of coating are being used today, but all processes require strict controls to maximize the effective use of silver. Today, conductive carbon fiber loaded ABS plastic is being used to reduce silver and eliminate stainless steel, brass, and nickel used in electrode components. These engineered resins reduce or eliminate the risk of burns in Magnetic Resonance Imaging (MRI) applications as well as corrosion or galvanic reactions. It has been suggested that biomonitoring sensors and electrodes of the future will employ the use of nano composites, microscopic circuitry, low power wireless, RFID, and other technologies.

The cost of the materials to construct disposable heart monitoring device 100 is relatively inexpensive and, therefore disposable heart monitoring device 100 is designed to be used once (or applied once to the chest for a period of time) and then discarded.

Each of the electrodes 104 comprises an output that is electrically coupled to a conductor, typically disposed within membrane 102. Each conductor terminates with signal collection unit 106. In another embodiment, one or more of the electrodes 104 utilizes RFID circuitry, or a similar, low-power, short range transmission technology, to transmit respective electrical signals to signal collection unit 106.

Signal collection unit 106 is shown as being located near a central point of membrane 102. However, in other embodiments, it may be located at different points. Signal collection unit 106 typically comprises a low-power A/D converter for converting the analog electronic signals from the electrodes into digital samples, then either storing the digital samples in a memory device and/or transmitting the digital samples to an external processing device, such as a smart phone or a personal computer, using well-known short-range, low power wireless transmission technology (e.g., Bluetooth, Wifi, RFID, etc.). Signal collection unit 106 may additionally comprise a connection port for transferring data via wire.

In one embodiment, each electrode 104 is affixed to the membrane 102 during the manufacturing process, and may be held in place by sewing the electrodes 104 into membrane 102, or by other well-known mechanical means. Alternatively, or in addition, in other embodiments, the electrodes 104 may be secured by an adhesive. In still other embodiments, the electrodes 104 are packaged separately from membrane 102, and a user installs each electrode onto membrane 102 just prior to use.

The location along membrane 102 of each of the electrodes is paramount to successful operation of disposable heart monitoring device 100. The size and shape of membrane 102 allows some of the electrodes 104 to be positioned over areas of the chest corresponding to the locations of electrodes during prior art heart monitoring procedures, such as an ECG procedure by a trained technician. However, in one embodiment, at least some of the electrodes 104 are placed in areas of the body that do not correspond with placement of electrodes in prior art heart monitoring procedures. For example, in an ECG procedure, electrodes known as peripheral electrodes, comprising RA (right arm), RL (right leg), LA (left arm) and LL (left leg) electrodes are placed on the aforementioned parts of the body. However, in embodiments of the present invention, these peripheral electrodes are positioned within/on membrane 102 such that they obtain electrical signals from the chest, rather than the limbs. This alternative placement enables a one-piece design, allowing users to more easily use the disposable heart monitoring device 100. As shown in FIG. 1, the RA electrode is shown as electrode 104a, the LA electrode is shown as electrode 104b the RL electrode is shown as electrode 104c, and the LL electrode is shown as electrode 104d.

Due to the alternative placement of the peripheral electrodes, the electrical signals that are generated by that make use of these electrodes may be different than what are produced by prior art ECGs. Therefore, adjustments to processing code may need to be made to account for these differences. For example, addition gain, attenuation and/or filtering may need to be added.

Returning now to proper placement of the electrodes on membrane 102, in one embodiment, six electrodes are positioned over areas of the chest corresponding to the locations of six chest electrodes in prior art ECG procedures. Electrode 104e is positioned on membrane 102 such that when membrane 102 is placed onto a user's chest, electrode 104e is positioned approximately over a fourth intercostal space at a right border of the sternum. Similarly, electrode 104g is positioned onto membrane 102 at a location corresponding to approximately the fourth intercostal space at a left border of the sternum when membrane 102 is placed onto a human chest. Electrode 104h is positioned on membrane 102 at a location approximately midway between the electrode 102f and electrode 102h. Electrode 102h is positioned on membrane 102 at a location corresponding approximately to at a mid-clavicular line in a fifth intercostal space when disposable heart monitoring device 100 is placed onto a human chest. Electrode 102i is positioned on membrane 102 at a location corresponding to an anterior axillary line approximately in the fifth intercostal space when disposable heart monitoring device 100 is placed onto a human chest. Finally, electrode 102j is positioned on membrane 102 at a location corresponding to approximately the mid-axillary line on the same horizontal level as the fourth electrode and the fifth electrode when the disposable heart monitoring device is placed onto a human chest.

The placement of the electrodes 104 in terms of distance between electrodes will vary depending on the size of membrane 102. For example, in a small version of disposable heart monitoring device 100, the electrodes 104 are spaced closer together than they would in a large version of disposable heart monitoring device 100.

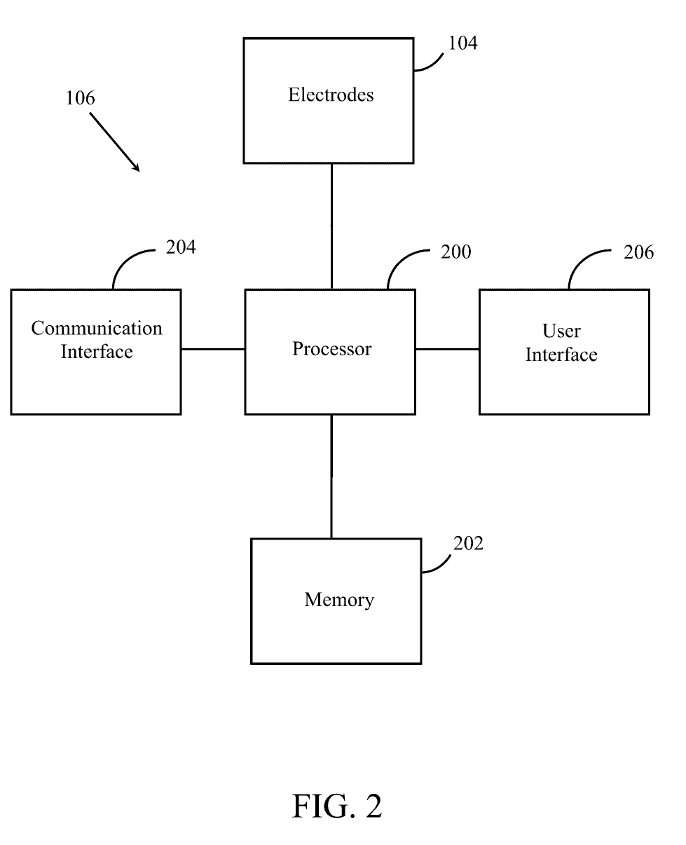

FIG. 2 is a functional block diagram of one embodiment of signal collection unit 106. Shown are processor 200, memory 202, communication interface 204, user interface 206, optional patient position device 208, and optional auxiliary port 210, as well as electrodes 104. It should be understood that the functional blocks may be coupled to each other differently in other embodiments and also that minor functional elements have been omitted for clarity (such as a battery). In this embodiment, processor 200 controls operation of signal collection unit 106, including conversion of analog signals from the electrodes into digital samples, storing the samples, and transmitting or otherwise providing the samples to an external processing device, in accordance with processor-executable instructions stored in memory 202. Processor 200 comprises one or more off-the-shelf microprocessors, microcontrollers, custom ASICs and is selected as having attributes of low power consumption, small size, and modest computing power.

Memory 202 comprises an information storage device such as RAM, SRAM, flash RAM, or forthcoming non-volatile memory technologies include FeRAM, CBRAM, PRAM, SONOS, RRAM, Racetrack memory, NRAM and Millipede. Memory 202 is used to store processor-executable code that, when executed by processor 200, causes disposable heart monitoring device 100 to convert analog signals from the electrodes into digital samples, potentially store the samples in memory 202, and transmit/provide the samples to an external processing device. The executable instructions may further comprise instructions that cause disposable heart monitoring device 100 to perform one or more self-tests, such as a test to determine if each of the electrodes are properly aligned, in good contact with a user's chest, or other tests. Memory 202 may also store sets of digital samples, each set corresponding to an ECG performed by a user of disposable heart monitoring device 100 over a period of time.

In one embodiment, at least some of memory 202 may be detachable from signal collection unit 106. In this embodiment, memory 202 comprises an insertable memory card, such as an SD, miniSD, or microSD memory card, thumb drive, etc., capable of being inserted and removed from signal collection unit 106. The memory 202 comprises enough memory to store multiple and/or continuous heart monitoring procedures over many hours, days or weeks, and may be removed after a number of procedures have been stored on it. Memory 202 may then be inserted into external processing device 302 and its contents used to generate heart monitoring results.

Communication interface 204 comprises circuitry to transmit or otherwise provide the digital samples to an external processing device, either as the digital samples are generated (e.g., real-time streaming) or from memory 202 after the digital samples have been stored. Communication interface typically comprises circuitry that consumes little power and is limited in range, such as Bluetooth, Wi-Fi, RFID, or some other well-known communication circuitry. In addition or alternatively, communication interface 204 comprises a port and circuitry to provide the digital samples via wire to an external processing device. In one embodiment, the port can be used to connect a portable, wireless communication device, such as a smart phone or personal computer, to signal collection unit 106 for wireless transmission of the digital samples to a remote location, such as a doctor's office, hospital, or other medical facility. Transmission by the wireless communication device may occur via cellular transmission or local data transmission via Bluetooth, Wi-fi, RFID, etc.

User interface 206 may comprise, simply, a multicolored LED and a pushbutton. Processor 200 may cause the multicolored LED to illuminate “green”, meaning that the disposable heart monitoring device 100 is ready to capture heart-related electronic signals. The user may press the pushbutton to begin the procedure, whereupon processor 200 may cause the LED to become extinguished, until the procedure is complete, at which time processor 200 causes the LED to illuminate green once again.

In other embodiments, user interface may comprise a series of LEDs or other low-power light-emitting devices to provide indications to a user as to an operating state of disposable heart monitoring device 100. In yet another embodiment, processor 200 provides detailed status information to an external processing device via communication interface 204 in order for the external processing device to display status information to the user, and for the user to use the external processing device to enter commands into disposable heart monitoring device 100. Such status may comprise “Ready”, “Testing”, a countdown time to indicate the completion of a procedure, “Self-test”, etc. Commands may comprise “Start Procedure” to begin recording electronic signals from the electrodes 104, “Perform calibration” to begin a calibration procedure, “Perform Self-Test”, and “Provide Data” to provide digital samples to the external processing device or wireless communication device.

Optional patient position device 208 comprises circuitry to detect a patient's orientation, e.g., whether the patient is lying down or standing, as knowledge of a patient's orientation may aid a physician in diagnosing and treating various disorders of the heart. For example, if an arrhythmia occurs while a patient is lying on his side, a treating physician may have the patient lie on his side in a follow-up appointment to quickly re-create the arrhythmia in order to better monitor and diagnose a condition, while minimizing the time needed to wait for the arrhythmia to re-present itself, saving valuable physician/technician time. Patient position device 208 could comprise a (MEMS) gyroscope, (MEMS) accelerometer, digital compass, or some other device capable of detecting a patient's position.

Optional auxiliary port 210 may be used to expand the functionality of disposable heart monitoring device 100 by enabling a connection to one or more peripheral devices. For example, connecting a pulse-monitoring wrist band or disposable finger-taped oximeter may allow disposable heart monitoring device 100 to act as a sleep apnea diagnosis device by receiving signals from such peripheral devices and evaluating them either alone or in combination with heart signals provided by electrodes 104. The signals from the oximeter and/or pulse-monitoring device are provided to eternal processing device 302 where they may be evaluated to determine a condition of sleep apnea, and/or they may be provided along with the electrical signals from the electrodes to remote processing facility 304 for similar analysis and diagnosis.

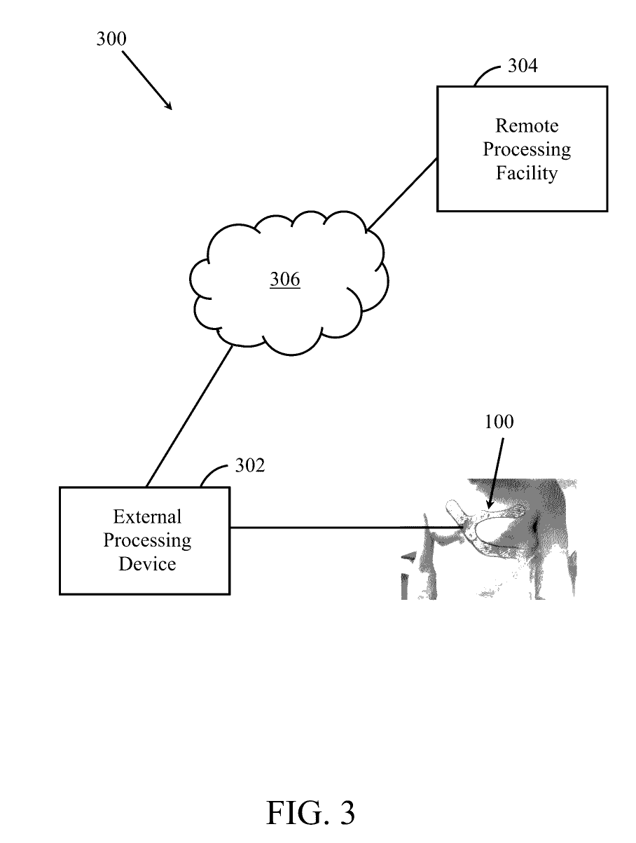

A sleep study generates several records of activity during several hours of sleep, usually about six. Generally, these records include an electroencephalogram, or EEG, measuring brain waves; an electroculogram, or EOG, measuring eye and chin movements that signal the different stages of sleep; an electrocardiogram, EKG, measuring heart rate and rhythm; chest bands that measure respiration; and additional monitors that sense oxygen and carbon dioxide levels in the blood and potentially record leg movement. Additional sensors/electrodes could be added to the membrane 102 and/or to port 210 to provide some or all of these additional monitoring functions, and the software stored in the external processing device 302 shown in FIG. 3 could be modified to provide this additional functionality.

FIG. 3 is an illustration of one embodiment of a system 300 for providing home heart monitoring. System 300 comprises disposable heart monitoring device 100, external processing device 302, and remote processing facility 304 coupled with external processing device 302 via one or more local area networks and/or wide-area networks 306. In this embodiment, external processing device 302 is used to generate ECG results to a user and/or to doctors or other health care professionals located at remote processing facility 304. Disposable heart monitoring device 100 provides digitized electronic signals that originate from electrodes 104 during an ECG procedure initiated by a user. The digitized electronic signals are provided to external processing device 302, where they may be stored in a memory and/or processed to produce the ECG results. Typically, external processing device 302 comprises a smart phone or personal computer that executes a software application for generating ECG results. In other embodiments, the software application performs additional functions, such as controlling operation of disposable heart monitoring device 100, transmitting the digitized electronic signals and/or ECG results to remote processing facility 304, and provides detailed information to a user of external processing device 302, such as displaying a history of ECG results over time, and/or warning a user about an abnormality in the digitized signals or the ECG results.

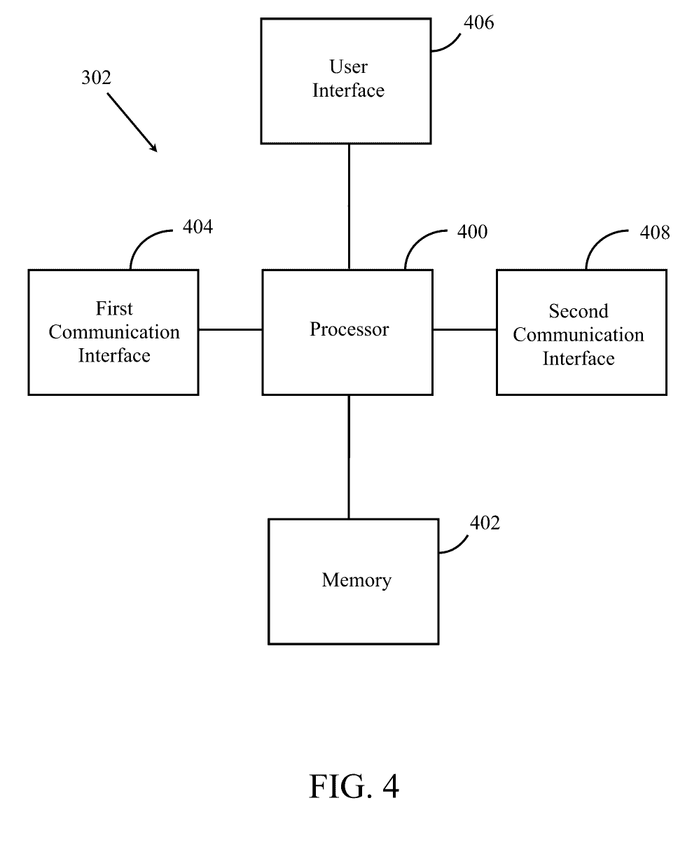

FIG. 4 is a functional block diagram of one embodiment of external processing device 302. Shown are processor 400, memory 402, first communication interface 404, user interface 406, and second communication interface 408. It should be understood that the functional blocks may be coupled to each other differently in other embodiments and also that minor functional elements have been omitted for clarity (such as a battery).

In this embodiment, processor 400 controls operation of external processing device 302, including receipt and sometimes storage of digitized electrical signals from disposable heart monitoring device 100, for computing ECG results from the digitized electrical signals, and for providing the digitized electrical signals and/or the ECG results to remote processing facility 304, as well as other functionality, as mentioned above. Processor 400 executes processor-executable instructions stored in memory 402 that causes external processing device 302 to perform the aforementioned functions. Processor 400 comprises one or more off-the-shelf microprocessors, microcontrollers, and/or custom ASICs, as is well known in the art.

Memory 402 comprises an information storage device such as RAM, SRAM, flash RAM, or forthcoming non-volatile memory technologies include FeRAM, CBRAM, PRAM, SONOS, RRAM, Racetrack memory, NRAM and Millipede. Memory 402 is used to store processor-executable code that, when executed by processor 400, causes external processing device 302 to perform the functions enumerated above. The executable instructions may further comprise instructions that cause external processing device 302 to perform other functions related to voice and data usage. Memory 402 may also store sets of digital samples, each set corresponding to an ECG performed by a user of disposable heart monitoring device 100 over a period of time. Such sets of digital samples may result from continuous monitoring of a user's heart over long periods of time, such as hours, days, weeks or longer.

First communication interface 404 comprises circuitry to receive digitized electronic signals from disposable heart monitoring device 100 and, in one embodiment, also transmit information or commands to disposable heart monitoring device 100. First communication interface typically comprises circuitry to support well-known, short-range communication protocols such as Bluetooth, Wi-Fi, RFID, etc. In addition or alternatively, first communication interface 404 comprises a port and circuitry to receive the digitized electrical signals via wire from disposable heart monitoring device 100.

User interface 406 typically comprises an electronic display screen and a combination of actual or virtual keyboards, microphones, pushbuttons, or other well-known means for entering information into external processing device 302.

Second communication interface 408 enables external processing device 302 to send and receive voice and/or data to/from remote devices and comprises well-known cellular and/or local-area network circuitry to perform those functions. Thus, the digitized electrical signals received from disposable heart monitoring device 100 may be transmitted to a remote location, such as remote processing facility 304. In one embodiment, the signals are encrypted to comply with regulatory mandates. Further, the ECG results that may be calculated by external processing device 302 may also be transmitted to remote processing facility 304 via second communication interface 408.

In one embodiment, the digitized electrical signals received from disposable heart monitoring device 100 and/or the ECG results calculated by external processing device 302 may be provided to remote processing facility 304 via first communication interface 404, if external processing device 302 is within range of a local-area network, such as a Wi-Fi network.

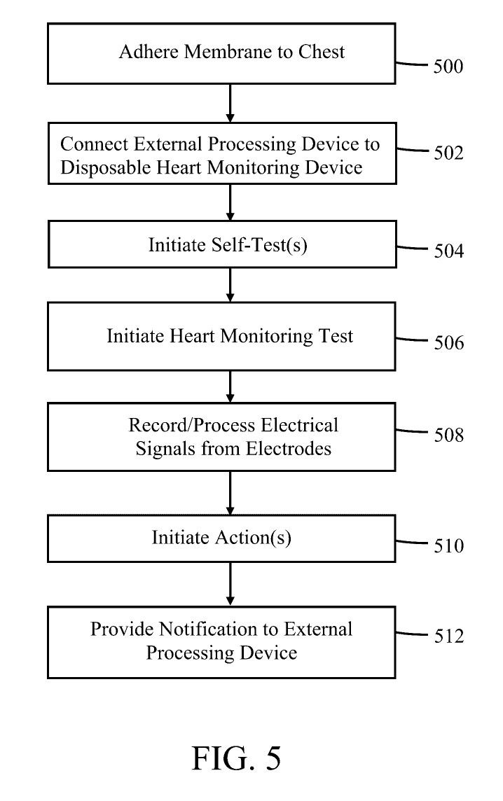

FIG. 5 is a flow diagram illustrating one embodiment of a method for providing home heart monitoring, performed by disposable heart monitoring device 100 and external processing device 302. It should be understood that in other embodiments, the order in which the functions are carried out may vary, and that some trivial functions have been omitted for clarity and brevity.

At block 500, a user of disposable heart monitoring device 100 adheres membrane 102 to the user's chest. Membrane 102 comprises an adhesive disposed on one side, allowing disposable heart monitoring device 100 to adhere to the user's chest. The user places membrane 102 on the user's chest such that the electrodes on/within membrane 102 are situated correctly over various parts of the chest needed to perform a 12-lead ECG. The peripheral locations where RA, RL, LA, and LL electrodes would normally be placed are not used. Rather, disposable heart monitoring device 100 incorporates these electrodes into membrane 102, where they are located at the very far ends of each strip that comprises membrane 102.

At block 502, in one embodiment, the user may couple external processing device 302 to disposable heart monitoring device 100 via wire or wireless means. In this embodiment, external processing device 302 is coupled to signal collection unit 106, where signal collection unit 106 acts as simply as “pass-through” of the signals provided by the electrodes 104. In one embodiment, however, signal collection unit 106 may comprise circuitry to pre-process the electrical signals generated by the electrodes 104. For example, signal collection unit 106 may comprise filtering components, an A/D converter, a processor and a memory to digitally pre-process the electrical signals, etc. In one embodiment, the functionality of disposable heart monitoring device 100 is initiated by external processing device 302. In another embodiment, functionality is initiated using a user interface on signal collection unit 106 and external processing device 302 used to receive and process the electrical signals. In other embodiments, a variety of combinations between functionality, data storage, data processing may be shared between external processing device 302 and signal collection unit 106. The remainder of this description will assume that these functions may be carried out on external processing device 302, signal collection unit 106, and/or a combination of both.

At block 504, after the membrane is put in place, the user may initiate a self-test to determine if the electrodes are properly located and/or orientated, to determine if each electrode is making proper contact with the user's skin, and to verify a patient position, e.g., whether the patient is in a horizontal or vertical position. The self-test may be initiated by a push of a button located on signal collection unit 106 or on external processing device 302 if signal collection unit 106 is coupled to external processing device 302. If the results of the self-test are negative, the user may be alerted via a sound or a light illuminating on signal collection unit 106 or on external processing device 302, or some other way to gain a user's attention. In one embodiment, the user may be alerted to which electrode(s) is(are) at fault, by analyzing the signals from each of the electrodes. In one embodiment, the self-test may be performed while signal collection unit 106 is in normal use by detecting any sudden loss or degradation of signal from one or more of the electrodes. In response to a negative self-test, the user may be prompted to move or reattach one or more electrodes, which may be called out by individually. For example, each of the electrodes could be numbered, and the user instructed to correct deficiencies associated with particular numbered ones of the electrodes. After re-positioning, the self-test may be run again, or run again only with respect to the electrode(s) that failed the self-test initially.

At block 506, a heart-monitoring procedure is initiated, either by the user or automatically. In an automatic embodiment, the executable code stored in either memory 202, 402, or both, causes either signal collection unit 106 and/or external processing device 302 to record the signals, in one embodiment, on a continuous basis or for predetermined time periods and/or repeated at predetermined time intervals. When it has been determined that the user is ready to begin the heart monitoring procedure, processing continues to block 508.

At block 508, the heart monitoring procedure begins. The electrical signals from the electrodes are monitored for a period of time, typically on the order of seconds, and used to perform calculations to assess heart functionality and determine whether any abnormalities exist. In one embodiment, the electrical signals are digitized using an A/D converter and the digitized samples used in conjunction with an algorithm stored in a memory to generate results, such as an ECG graphical representation of the user's heart functionality. Other results may be calculated as well, such as tabulated data relating to the user's heart function. For example, a standard 12-lead ECG report shows a 2.5 second tracing of each of the twelve leads. The tracings are most commonly arranged in a grid of four columns and three rows. The first column is the limb leads (I, II, and III), the second column is the augmented limb leads (aVR, aVL, and aVF), and the last two columns are the precordial leads (V1-V6). In one embodiment, results are calculated “on the fly” as the electrical signals are received. This may involve temporary storage of the electrical signals. In another embodiment, the electrical signals are stored in memory, then accessed at a later time to perform the calculations.

The algorithm used to calculate results utilize algorithms similar to what is used to generate results in prior art heart-monitoring machines. In one embodiment, however, because the RA, RL, LA, and LL electrodes are positioned at different parts of the body (i.e., all on the user's chest), adjustments to the algorithm are made to account for the different electrical readings that are produced during a heart-monitoring procedure as a result of the placement of the RA, RL, LA, and LL electrodes. Such adjustments may comprise an increase in gain and/or change in filtering characteristics of the signals received.

After the heart monitoring procedure has been completed, a number of actions may be taken, shown as block 510. In one embodiment, where the results of the heart-monitoring procedure are displayed to the user, along with any tabulated data that may have been produced. For example, in an ECG, graphical representations of heart function may be displayed to the user, and a table of results also provided. The user may initiate an action to transmit the results and/or the electrical signals to remote processing facility 106. In another embodiment, the electrical signals are automatically provided to remote processing facility 106. The user typically may be permitted to view the results if desired.

At block 512, a doctor or other medical personnel may evaluate the results of one or more heart monitoring tests performed by the user, and determine that a course of action should follow, depending on the results. For example, if one or more test results indicate an immediate cause of concern, then the doctor or other medical personnel may contact the user via traditional phone, text, or email to inform the user of the immediate cause of concern, and to have the user seek immediate medical attention. In another embodiment, a notification is sent from remote processing facility 106 to external processing device 302, informing the user of one or more results of the heart monitoring procedures, and whether the results were normal or not, and may further comprise instructions for the user to follow, or a recommendation for the user to seek medical attention.

The previous description of the preferred embodiments is provided to enable any person skilled in the art to make and use the concepts described herein. The various modifications to these embodiments will be readily apparent to those skilled in the art, and the generic principles defined herein may be applied to other embodiments without the use of the inventive faculty. Thus, the ideas presented are not intended to be limited only to the embodiments discussed herein, but are to be accorded the widest scope consistent with the principles and novel features disclosed herein.

I claim:

1. A disposable heart monitoring device, comprising:
a membrane comprising an adhesive surface for adhering the membrane to a human chest;
a first plurality of electrodes attached to the membrane at locations corresponding to chest electrode positions of a standard heart monitoring procedure;
a second plurality of electrodes attached to the membrane corresponding to peripheral electrodes of a standard heart monitoring procedure for placement against a human chest;
a signal collection unit coupled to the first plurality of electrodes and the second plurality of electrodes for receiving electrical signals from the first plurality of electrodes and the second plurality of electrodes; and
means for providing the electrical signals to a processing device separate from the disposable heart monitoring device for analyzing the electrical signals to evaluate one or more attributes of a human heart.

2. The disposable heart monitoring device of claim 1, wherein the membrane further comprises:
an elongated, curved strip having a first end for placement over an upper right chest location;
a middle portion for placement across a sternum;
a second end for placement over a lower left chest location,
a short, lower strip extending away from the elongated, curved strip at an acute angle from the second end directed downward when the membrane is attached to a human chest;
and a mid-strip extending approximately horizontally from the elongated, curved strip for placement over an upper, left location of a human chest.

3. The disposable heart monitoring device of claim 1, wherein the first plurality of electrodes comprise:
a first electrode positioned approximately on the membrane at a point corresponding to a fourth intercostal space at a right border of the sternum when the disposable heart monitoring device is placed onto a human chest;
a second electrode positioned approximately on the membrane at a location corresponding to the fourth intercostal space at a left border of the sternum when the disposable heart monitoring device is placed onto a human chest;
a third electrode positioned approximately on the membrane at a location approximately midway between the second electrode and a fourth electrode when the disposable heart monitoring device is placed onto a human chest;
the fourth electrode positioned approximately on the membrane at a location corresponding to at a mid-clavicular line in a fifth intercostal space when the disposable heart monitoring device is placed onto a human chest;
a fifth electrode positioned approximately on the membrane at a location corresponding to an anterior axillary line in the fifth intercostal space when the disposable heart monitoring device is placed onto a human chest; and
a sixth electrode positioned approximately on the membrane at a location corresponding to the mid-axillary line on the same horizontal level as the fourth electrode and the fifth electrode when the disposable heart monitoring device is placed onto a human chest.

4. The disposable heart monitoring device of claim 1, wherein one of the plurality of second electrodes is located on the membrane at the first end and corresponds to an RA electrode of a standard ECG, located on a right arm.

5. The disposable heart monitoring device of claim 1, wherein one of the plurality of second electrodes is located on the membrane at an end of the short, lower strip furthest away from the elongated, curved strip and corresponds to an RL electrode of a standard ECG, located on a right leg.

6. The disposable heart monitoring device of claim 1, wherein one of the plurality of second electrodes is located on the membrane at an end of the mid-strip furthest away from the elongated, curved strip and corresponds to an LA electrode of a standard ECG, located on a left arm.

7. The disposable heart monitoring device of claim 1, wherein one of the plurality of second electrodes is located on the membrane at the second end and corresponds to an LL electrode of a standard ECG, located on a left leg.

8. The disposable heart monitoring device of claim 1, wherein the number electrodes and the number of second electrodes enables a 12-lead electrocardiogram to be generated by the external processing device.

9. The disposable heart monitoring device of claim 1, wherein the number electrodes and the number of second electrodes enables a 48-lead electrocardiogram to be generated by the external processing device.

10. The disposable heart monitoring device of claim 1, wherein the first plurality of electrodes comprises six electrodes and the second plurality of peripheral leads comprises four electrodes.

11. The disposable heart monitoring device of claim 1, wherein the signal collection unit comprises a processor for performing a self-test to determine whether the electrodes are properly placed against a human chest.

12. The disposable heart monitoring device of claim 1, further comprising a position-determination device for determining an orientation of a user of the disposable heart monitoring device.

13. The disposable heart monitoring device of claim 1, wherein the signal collection unit comprises a memory, wherein the memory is capable of storing multiple sets of electrical signals corresponding to multiple ECG readings taken over time.

14. The disposable heart monitoring device of claim 1, further comprising a battery for powering the signal collection unit.

15. The disposable heart monitoring device of claim 1, further comprising:
a port for providing wired communication with a wireless transmission unit; and
the wireless transmission unit for coupling with the disposable heart monitoring device via the port, and for wirelessly transmitting the electrical signals to the analysis device.

16. The disposable heart monitoring device of claim 15, further comprising:
a memory for storing multiple sets of the electrical signals corresponding to multiple ECG readings taken over time.

17. The disposable heart monitoring device of claim 1, further comprising:
an auxiliary port for receiving signals from an oximeter, wherein the means for providing the electrical signals to a processing device separate from the disposable heart monitoring device additionally provide the signals from the oximeter to evaluate a condition of sleep apnea

18. A system for providing home heart monitoring, comprising:
a disposable membrane comprising:
an adhesive surface for adhering the membrane to a human chest;
a plurality of electrodes; and
a signal collection unit coupled to the plurality of electrodes for receiving electrical signals from the plurality of electrodes and for providing the electrical signals to an external processing device separate from the disposable heart monitoring device for analyzing the electrical signals to evaluate one or more attributes of a human heart;
and
the external processing device, comprising:
means for receiving the electrical signals from the signal collection unit;
a memory for storing the electrical signals and processor-executable instructions; and
a processor for executing the processor-executable instructions that cause the external processing device to:
compute one or more graphs of the electrical signals vs. time;
store the one or more graphs in the memory; and
transmit the one or more graphs to a remote location.

19. The system of claim 18, wherein the membrane further comprises an elongated, curved strip having a first end for placement over an upper right chest location, a middle portion for placement across a sternum, and a second end for placement over a lower left chest location, the elongated, curved strip further comprising a short, lower strip extending at an acute angle from the second end directed downward when the membrane is attached to a human chest, and a mid-strip extending approximately horizontally from the elongated, curved strip for placement over an upper, left location of a human chest.

20. The system of claim 18, wherein at least some of the electrodes provide electrical signals corresponding to peripheral electrodes of a standard heart monitoring procedure.
